(19) United States
(12) Patent Application Publication
(10) Pub. No.: US 2007/0116778 A1
(43) Pub. Date: May 24, 2007

(54) Antibacterial, antiviral, and antifungal nutritional supplement: a combination of natural or neutraceutical compounds shown to have antibacterial, antiviral, and antifungal properties, to help boost the immune system in humans, and to protect against certain biowarfare diseases

(76) Inventor: Arthur A. Fierro, Harrisonburg, VA (US)

Correspondence Address: DR ARTHUR A. FIERRO, 620 NEFF AVE, HARRISONBURG, VA, 22801, US

(21) Appl. No.: 11/590,552

(22) Filed: Nov. 1, 2006

Related U.S. Application Data
(60) Provisional application No. 60/597,310, filed on Nov. 22, 2005.

(51) Int. Cl. A61K 38/05; A61K 31/715; A61K 31/66; A61K 31/385; A61K 35/20; A61K 31/20; A61K 36/71; A61K 33/32; A61K 36/58

(52) U.S. Cl. 424/535; 424/641; 424/726; 424/761; 514/18; 514/54; 514/102; 514/558; 514/562; 514/440; 514/731

(57) ABSTRACT

Certain compounds have been shown to provide antibacterial, antiviral, and antifungal properties. These compounds can provide some of the beneficial aspects of medicinal antibiotics without damaging or destroying beneficial bacteria in the human body. By combining these compounds into a single product in capsule form, an effective wide-spectrum natural ‘antibiotic’ can be produced, without the negative side effects of medicinal antibiotics. This product can destroy harmful bacteria, viruses, and fungi without eliminating beneficial bacteria or creating medicine-resistant strains.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING

Not Applicable

### BACKGROUND OF THE INVENTION

U.S. Provisional Patent Application No. 60/597,310. Filing date of U.S. Provisional Patent Nov. 22, 2005

The present invention relates to an over-the-counter supplement that is a natural antibiotic, antiviral, antifungal for human consumption, to raise the immune system and protect against certain bioterrorist diseases.

Antibiotic literally means against (anti) life (biotic). Antibiotic therapy has limited specificity and generally fails to discriminate beneficial bacteria from pathogenic bacteria. The result is to possibly destroy all bacteria. This can lead to decolonization of beneficial bacteria in the human gut. Destruction of beneficial bacteria can lead to adverse health conditions such as irritable bowel syndrome, yeast and fungal overgrowth. It is often at this point that additional antibiotic therapy is introduced. The repetitious use of antibiotic therapy, while destroying weaker bacteria, allows the stronger, more “resistant” bacteria to thrive. We have come to a point in history that super virulent bacteria are now in existence that do not respond to conventional antibiotic treatment. This is exemplified in the concern of bioterrorist pathogens such as anthrax. The Centers for Disease Control (CDC) advised the medical field in 2003 that the conventional antibiotic used against Anthrax was no longer effective.

Bacteria defend against antibiotic therapy in a number of ways. They can mutate their entry pores (porins). They can secrete an enzyme that destroy the antibiotic's “lactam ring”; suppressing transpeptidase, which could stop the bacteria's ability to replicate itself. Bacteria can even mutate their own transpeptidase so that the antibiotic cannot bond to it.

Viruses must use a host to survive as they lack ribosomes necessary for replication. By using a host's ribosomes, viruses also use the host's energy and amino acids. Viruses have protective adaptations to help ensure their survival. Viruses have a protein coat (capsid) that surrounds their genetic material. This is additionally protected by an envelope of fat. The structure is enclosed by an outer protective membrane made up of glycoprotein and fatty acids. Currently there are very few antiviral medications that have been shown to be effective in destroying viruses.

Fungi, which include molds, mushrooms and yeast, are parasitic life forms. Pathogenic fungi are usually kept in check by sufficient quantities of beneficial bacteria, hydrochloric acid and bile salts. Fungal infections may mutate to a yeast form, such as candidiasis. Fungal medications are somewhat effective against fungi, but do not address mutation to a yeast state, and have undesirable side effects such as intestinal distress.

Research on each of the following compounds, put into one product, has shown that there is a commonality of pathogenic bacteria, viruses and fungi. Each appears to have the ability to mutate, each use iron as their energy, and each protect their ability to replicate. Research has also shown that while pathogenic organisms use iron as a fuel source, beneficial organisms do not use iron.

Monolaurin, a medium chain triglyceride was first identified in the 1970's. Initial clinical trials to test the effectiveness of Monolaurin on viruses started with 15 HIV infected patients at the San Lazaro Hospital in Manila under Dr. Eric Tayag. Patients were randomly placed into 3 treatment groups each receiving a different dosage of Monolaurin as coconut oil: 7.2 gML, 2.4 gML and 50 ML. This daily regime of 6 months duration was clinically evaluated by laboratory testing consisting of CD4 and CDS counts, CBC, blood lipids, liver and kidney functions initially and at 3 and 6 months. By the 6th month, 8 patients showed a reduced viral load without any side effects. A series of papers published in the 1970s supported the effectiveness of Monolaurin against viruses, bacteria and fungi. Monolaurin was then classified by the USDA as GRAS.

Since the initial studies performed in the 1970s clinical trials have continued to support the properties of Monolaurin. In Vitro Susceptibilities of Neisseria gonorrhoae to Fatty Acids and monoglycerides Gudmundur Bergsson, Olafgur Steingrison, and Halldor Thormar Antimicrobial Agents and Chemotherapy, November 1999, p. 2790-2792, Vol. 43, No. 11. This article showed how Monolaurin dissolves the outer lipid membrane and interferes with transcriptinase production of this pathogen. Similar findings were reported: In vitro inactivation of Chlamydia trachomatis by fatty acids and monoglycerides. Antimicrobial Agents Chemotherapy. 1998 Bergsson, G., O. Steingrimsson, and H. thormar. Icelandic Medical Journal No 84 Supplement 37, p. 118. sacs, C. E., R. E. Litov, and H. Thormar 1994 Inactivation of enveloped viruses in human milk, infant formula, and bovine milk. J. Nutritional Biochemistry 6:362-366.

Oleuropein, or Extract of Olive Leaf has been extensively studied for its broad antimicrobial properties. Oleuropein and derivatives have been shown to inhibit or delay the rate of growth of a range of bacteria and microfungi: J. Pharm Pharmacol. 1999 August; 51(8):971-4: On the in-vitro antimicrobial activity of oleuropein and hydroxytryrosol. Bisignano, G., Tomaino, A., Lo Cascio R., Crisafi G., Uccella N., Saija A.: Laboratory testing by Thome Research: In vitro susceptibility test indicates that Olive Leaf Extract is an effective post exposure prophylaxis against Bacillus anthracis and Yesinia Pestus

IP6 A commonality of pathogenic viruses, bacteria and fungi are their use of iron as their source of energy. Inhibiting the viruses, bacteria and fungus ability to use iron, effectively starves their ability to survive. IP6 by blocking iron prevents cell signaling and prevents replication. Berridge, M. J, Irvine, R. F. Inositol phosphates and cell signaling: 1987: Nature: 314: 197-205.

Beta 1, 3 Glucan is extracted from purified yeast, with the protein antigens removed, avoiding human sensitivity reactions. Beta 1,3 Glucan in the presence of foreign material in the human body, acts as the “switch” triggering the release of interferon that signals the immune system (macrophages) to mobilize against disease causing bacteria, viruses, and fungi. Czopjk, Austin, K. F.: A b-glucan inhabitable receptor on human monocytes; its identity with phagocytic receptor for particular activators of alternate complement pathways. J. Immunology 1985: 134: 2588-2593.

Colostrum is a component of mother's milk or “first milk”. Among its immunological properties is that it delivers the immunoglobulins IgG, IgA, IgE, IgM. These immunoglobulin are the proteins that are necessary to building our immune system. In addition, colostrum provides interferon, the “switch” that, when activated by Beta 1, 3, Glucan, mobilizes the macrophages to attack pathogenic bacteria, viruses, and fungi.

MSM is found in mother's milk, fruits and vegetable. In the protocol to destroy pathogenic bacteria, viruses and fungi, MSM provides natural sulfur which protects the white blood cells. MSM performs that as the specific antioxidant with an affinity for the white blood cells. The antioxidant activity helps to prevent the oxidation and subsequent breakdown of the white Herschler, R. J.: Methysulfonylmethane and Methods of use. American Academy of Science. 1983 (411).

Secondary compounds consisting of: Echinacea Purpurea, garlic, golden seal and Neem Leaf Extract all have antibiotic, antiviral and antifungal properties.

Alpha Lipoic Acid, L-Glutathione and N-Acetyl Cysteine are all antioxidants that serve to protect human body cells from oxidation, or breakdown from foreign material such as viruses, bacteria and fungal agents.

### BRIEF SUMMARY OF THE INVENTION

Invention is a unique combination of neutraceutical compounds that acts as a natural antibiotic, antiviral, antifungal supplement. Its ingredients destroy the outer membrane of pathogenic bacteria, viruses and fungi, cleave their DNA, starve their food source and at the same time protect and build our immune system.

The invention, as a supplement, contains the following components:

Monolaurin—dissolves the outer protective layer of pathogenic organisms.

Oleuropein (Extract of Olive Leaf)—splits or cleaves the DNA structure.

IP6 bonds onto Iron rendering it unavailable as an energy source.

Beta 1,3 Glucan—activates the immune system.

Colostrum—supplies immunoglobulins and interferon.

Calcium Phytate—raises the body pH and acts as a substrate for new DNA synthesis.

Secondary components: zinc, MSM, DMG, phosphorus, alpha Lipoic acid, N-acetyl-cysteine, L-Glutathione.

Proprietary blend: Echinacea pupurea, garlic, golden seal, and Neem Leaf extract.

1. The invention composed of the combination of Monolaurin, Oleuropein, IP6, Beta 1, 3 Glucan, Colostrum, MSM, Calcium Phytate, Alpha Lipoic Acid, N-Acetyl-cysteine, Echinacea, Zinc, DMG, Glutathione, Neem Leaf Extract and Golden Seal.
